(19) United States
(12) Patent Application Publication
(10) Pub. No.: US 2013/0017572 A1
(43) Pub. Date: Jan. 17, 2013

(54) CODON MODIFIED POLYNUCLEOTIDE SEQUENCES FOR ENHANCED EXPRESSION IN A HOST SYSTEM

(71) Applicants: Peter S. Lu, Fremont, CA (US); Johannes Schweizer, Fremont, CA (US); Chamorro Somoza Diaz-Sarmiento, Mountain View, CA (US)

(72) Inventors: Peter S. Lu, Fremont, CA (US); Johannes Schweizer, Fremont, CA (US); Chamorro Somoza Diaz-Sarmiento, Mountain View, CA (US)

(21) Appl. No.: 12/919,684

(22) PCT Filed: Apr. 30, 2010
(86) PCT No.: PCT/US10/33309

§ 371 Date: Aug. 26, 2010

Related U.S. Application Data
(60) Provisional application No. 61/174,462, filed on Apr. 30, 2009.

(51) Int. Cl. C12N 15/34; C12P 21/00

(52) U.S. Cl. 435/69.3; 536/23.72

(57) ABSTRACT

Synthetic DNA molecules encoding various HPV proteins are provided. The codons of the synthetic molecules are designed so as to use the codons that preferentially increase expression of the polypeptide in the host cell, which in preferred embodiments is a human cell. The codons are modified in order to minimize, decrease or eliminate cellular destruction of the polypeptide construct.

### CROSS REFERENCE

This application claims the benefits of U.S. Provisional Application No. 61/174,462, filed Apr. 30, 2009, which is incorporated by reference herein in its entirely.

### BACKGROUND OF THE INVENTION

Cervical cancer is the second most common cancer diagnosis in women and is linked to high-risk human papillomavirus infection 99.7% of the time. Currently, 12,000 new cases of invasive cervical cancer are diagnosed in US women annually, resulting in 5,000 deaths each year. Furthermore, there are approximately 400,000 cases of cervical cancer and close to 200,000 deaths annually worldwide. Human papillomaviruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

Papillomavirus infections occur in a variety of animals, including humans, sheep, dogs, cats, rabbits, snakes, monkeys and cows. Papillomaviruses infect epithelial cells, generally inducing benign epithelial or fibroepithelial tumors at the site of infection. Papillomaviruses are species specific infective agents; a human papillomavirus cannot infect a non-human.

A number of lines of evidence point to HPV infections as the etiological agents of cervical cancers. Papilloma viruses have a DNA genome which encodes “early” and “late” genes designated E1 to E7, L1 and L2. The early gene sequences have been shown to have functions relating to viral DNA replication and transcription, evasion of host immunity, and alteration of the normal host cell cycle and other processes. For example the E1 protein is an ATP-dependent DNA helicase and is involved in initiation of the viral DNA replication process whilst E2 is a regulatory protein controlling both viral gene expression and DNA replication. Through its ability to bind to both E1 and the viral origin of replication, E2 brings about a local concentration of E1 at the origin, thus stimulating the initiation of viral DNA replication. The E4 protein appears to have a number of poorly defined functions but amongst these may be binding to the host cell cytoskeleton, whilst E5 appears to delay acidification of endosomes resulting in increased expression of EGF receptor at the cell surface and both E6 and E7 are known to bind cell proteins p53 and pRB respectively. The E6 and E7 proteins form HPV types associated with cervical cancer are known oncogenes. L1 and L2 encode the two viral structural (capsid) proteins. Multiple studies in the 1980's reported the presence of HPV variants in cervical dysplasias, cancer, and in cell lines derived from cervical cancer. Further research demonstrated that the E6-E7 region of the genome from oncogenic HPV 18 is selectively retained in cervical cancer cells, suggesting that HPV infection could be causative and that continued expression of the E6-E7 region is required for maintenance of the immortalized or cancerous state. The following year, Sedman et al demonstrated that the E6-E7 genes from HPV 16 were sufficient to immortalize human keratinocytes in culture. Barbosa et al demonstrated that although E6-E7 genes from high risk HPVs could transform cell lines, the E6-E7 regions from low risk, or non-oncogenic variants such as HPV 6 and HPV 11 were unable to transform human keratinocytes. More recently, Pillai et al examined HPV 16 and 18 infection by in situ hybridization and E6 protein expression by immunocytochemistry in 623 cervical tissue samples at various stages of tumor progression and found a significant correlation between histological abnormality and HPV infection.

The majority of genital warts (>90%) contain HPV genotypes 6 and 11. Whilst HPV-6 is the most prevalent genotype identified in single infections, both HPV-6 and HPV-11 may occasionally occur in the same lesion. Warts generally occur in several sites in infected individuals and more than 60% of patients with partners having condyloma (genital warts) develop lesions, with an average incubation time of 3 months. A range of treatment options are currently available. However, they rely upon excision or ablation and/or the use of topical gels and creams. They arc not pain free, they may require frequent clinic visits, and efficacy is highly variable. Disease recurrence remains a significant problem for the effective management of this disease.

HPV has proven difficult to grow in tissue culture, so there is no traditional live or attenuated viral vaccine. Development of an HPV vaccine has also been slowed by the lack of a suitable animal model in which the human virus can be studied. This is because the viruses arc highly species specific, so it is not possible to infect an immunocompetent animal with a human papilloma virus, as would be required for safety testing before a vaccine was first tried in humans.

The detection and diagnosis of disease is a prerequisite for the treatment of disease. Numerous markers and characteristics of diseases have been identified and many are used for the diagnosis of disease. Many diseases are preceded by, and are characterized by, changes in the state of the affected cells. Changes can include the expression of pathogen genes or proteins in infected cells, changes in the expression patterns of genes or proteins in affected cells, and changes in cell morphology. The detection, diagnosis, and monitoring of diseases can be aided by the accurate assessment of these changes. Inexpensive, rapid, early and accurate detection of pathogens can allow treatment and prevention of diseases that range in effect from discomfort to death.

Retooling coding regions encoding polypeptides using codon frequencies preferred in a given mammalian species has been used to increase expression of the polypeptide in the cells of that mammalian species. See, e.g., Deml, L., et al., J. Virol. 75:10991-11001 (2001), and Narum, D L, et al., Infect. Tmmun. 69:7250-7253 (2001), all of which are herein incorporated by reference in its entirety. However, many polypeptides, although codon optimized for a particular cell line, still have little or no polypeptide expression.

There remains a need in the art for methods and compositions that can increase the expression of polypeptides in different cell lines.

### SUMMARY OF THE INVENTION

The present invention encompasses a method comprising modifying a nucleic acid molecule, wherein the nucleic acid molecule comprises a sequence of nucleotides that is codon-modified for high level expression in a host cell.

The present invention further encompasses a method comprising modifying a nucleic acid molecule, wherein the nucleic acid molecule comprises a sequence of nucleotides that is codon-modified for high level expression in a host cell, transforming a host cell with the nucleic acid molecule; and cultivating the transformed cell under conditions that permit expression of the nucleic acid molecule to produce a protein product. The present invention also encompasses compositions produced by the methods described. In one embodiment, the nucleic acid molecule has been modified by at least 10% from the native sequence. In another embodiment, the nucleic acid molecule has been modified such that at least 10% of the codons have been modified. In another embodiment, the nucleic acid molecule has been modified such that at least 5% of the codons have the maximum number of changes such that there is still degeneracy for the amino acid originally encoded. In another embodiment, the nucleic acid molecule has been modified such that at least 5% of the codons have been modified to have a ration of usage less than 1. In another embodiment, the nucleic acid molecule codes for a human papilloma virus E6.

In another embodiment, the present invention is a method comprising the steps of: (a) Modifying a nucleic acid molecule, wherein the nucleic acid molecule comprises a sequence of nucleotides that is codon-modified for high level expression in a host cell; (b) transforming a host cell with the nucleic acid molecule; and (c) cultivating the transformed cell under conditions that permit expression of the nucleic acid molecule to produce a protein product. In another embodiment, the nucleic acid molecule has been modified by at least 10% from the native sequence. In another embodiment, the nucleic acid molecule has been modified such that at least 10% of the codons have been modified. In another embodiment, the nucleic acid molecule has been modified such that at least 5% of the codons have the maximum number of changes such that there is still degeneracy for the amino acid originally encoded. In another embodiment, the nucleic acid molecule has been modified such that at least 5% of the codons have been modified to have a ration of usage less than 1. In another embodiment, the nucleic acid molecule codes for human papilloma virus E6. In another embodiment, the host cell is a 293-HEK or C33A cell.

In another embodiment, the present invention is a composition comprising a modified nucleic acid molecule. In another embodiment, the nucleic acid molecule has been modified by at least 10% from the native sequence. In another embodiment, the nucleic acid molecule has been modified such that at least 10% of the codons have been modified. Tn another embodiment, the nucleic acid molecule has been modified such that at least 5% of the codons have the maximum number of changes such that there is still degeneracy for the amino acid originally encoded. In another embodiment, the nucleic acid molecule has been modified such that at least 5% of the codons have been modified to have a ration of usage less than 1.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a nucleic acid sequences comparison of HPV35-E6 wild type sequence and codon optimized sequence towards human codon preference.

FIG. 2 shows a nucleic acid sequences comparison of HPV35-E6 wild type sequence and codon modified sequence towards maximum distance to the viral E6 gene sequence.

FIG. 3 shows the amino acid sequence coded by both the codon optimized and codon modified sequences of FIGS. 1 and 2.

FIG. 4 shows a Western Blot of a stably transfected cell line expressing HPV35-E6 using the codon modified sequence.

### DETAILED DESCRIPTION OF THE INVENTION

Synthetic DNA molecules encoding various HPV proteins are provided. The codons of the synthetic molecules are designed so as to use the codons that preferentially increase expression of the polypeptide in the host cell, which in preferred embodiments is a human cell. In preferred embodiments, the codons are modified in order to minimize, decrease or eliminate cellular destruction of the polypeptide construct. This differs from conventional methods in that it seeks not to mimic a host cell's native codon usage, but differentiates from the native codon sequence of the transfected polypeptide. The synthetic molecules may be used to generate the polypeptide of the polynucleotide sequence or use the transfected cell, for example, to screen for candidate diagnostic or therapeutic candidates, or as a polynucleotide vaccine which provides effective immunoprophylaxis against papillomavirus infection through neutralizing antibody and cell-mediated immunity The synthetic molecules may be used as an immunogenic composition. This invention provides polynucleotides which, when directly introduced into a vertebrate in vivo, including mammals such as primates and humans, or in vitro, including human cell lines, induce the expression of encoded proteins within the animal or cell.

The gene encoding a polypeptide, for example E6 from any serotype HPV, can be modified in accordance with this invention. It is preferred that the nucleotide sequence chosen be one which is known to produce low polypeptide expression in the host cell. This may be due to host cell recognition of the polynucleotide sequence as foreign. Examples of polynucleotides for transfection include, but are not limited to, E6 polynucleotide from the HPV strains: HPV6a, HPV6b, HPV11, HPV16, HPV18, HPV31, HPV33, HPV35, HPV39, HPV45, HPV51, HPV52, HPV56, HPV58, HPV68 or variants thereof.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The term “analogue” refers to a polynucleotide which encodes the same amino acid sequence as another polynucleotide of the present invention but which, through the redundancy of the genetic code, has a different nucleotide sequence whilst maintaining the same codon usage pattern, for example having the same codon usage coefficient or a codon usage coefficient within 0.1, preferably within 0.05 of that of the other polynucleotide.

The term “codon usage pattern” refers to the average frequencies for all codons in the nucleotide sequence, gene or class of genes under discussion (e.g. highly expressed mammalian genes). Codon usage patterns for mammals, including humans can be found in the literature (see e.g. Nakamura et. al. Nucleic Acids Research 1996, 24:214 215).

In the codon optimization methods, the codon usage pattern is altered from that typical of human papilloma viruses to more closely represent the codon bias of the target organism, e.g. E. coli or a mammal, especially a human. The “codon usage coefficient” is a measure of how closely the codon usage pattern of a given polynucleotide sequence resembles that of a target species. Codon frequencies can be derived from literature sources for the highly expressed genes of many species (see e.g. Nakamura et. al. Nucleic Acids Research 1996, 24:214 215). The codon frequencies for each of the 61 codons (expressed as the number of occurrences occurrence per 1000 codons of the selected class of genes) are normalized for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

In the polynucleotides and methods of the present invention, the codon usage pattern is altered from that typical of human papilloma viruses to modify the codons without altering the coded amino acid sequence. The methods of “codon modification” differ from codon optimization. In codon optimization, the sequences are modified to most closely mimic the codon usage in the native cells. In codon modification, the sequences are modified to maximally differ from the original wildtype polynucleotide sequence while maintaining the codon degeneracy to code for the polypeptide.

Shorter polynucleotide sequences are within the scope of the invention. For example, a polynucleotide of the invention may encode a fragment of a HPV protein. A polynucleotide which encodes a fragment of at least 8, for example 8 or 10 amino acids or up to 20, 50, 60, 70, 80, 100, 150 or 200 amino acids in length is considered to fall within the scope of the invention as long as the polynucleotide has a codon usage pattern which resembles that of a highly expressed mammalian gene and the encoded oligo or polypeptide demonstrates HPV antigenicity. In particular, but not exclusively, this aspect of the invention encompasses the situation when the polynucleotide encodes a fragment of a complete HPV protein sequence and may represent one or more discrete epitopes of that protein.

The polynucleotides of the present invention show higher expression in particular cell lines (e.g. C33A) than corresponding wild-type sequences encoding the same amino acid sequences. Whilst not wishing to be bound by any theory, this is believed to be due to cellular recognition of foreign polynucleotide sequences. By altering the polynucleotide sequence through codon modification, the host cell does not recognize the sequence as a foreign threat while maintaining the coding information for the polypeptide.

Codon modification, herein referred to as “codon modification” or “codon modified” refers to the alteration of gene sequences such that codons are replaced with degenerate codons that code for the same amino acid. This differs from codon optimization, a process well known in the art, in that codon modification seeks to use degenerate codons that are used less frequently in the host cell or animal than another degenerate codon coding for the same amino acid. Without being limited by theory, codon optimization seeks to increase the efficiency of translation by using machinery ideally suited within a host cell for producing proteins by using common codons for amino acids that are likely to have higher concentrations of tRNA molecules to build the protein. Codon modification seeks to generate sequences that differ from the normal sequences seen in the host cell in order to evade degradation mechanisms within a host cell.

Codon Modification for HPV E6 Polynucleotides

The wild-type sequences for many HPV E6 genes are known. In accordance with this invention, HPV gene segments were converted to sequences having identical translated sequences but with alternative codon usage. The methodology may be summarized as follows:

1. Identify placement of codons for proper open reading frame.

2. Compare wild type codon and degenerate codons that code for the same amino acid.

3. Replace codon with different degenerate codon, preferably degenerate codon with the greatest variability from the host preferred codon.

4. Repeat this procedure until the entire gene segment has been replaced.

5. Inspect new gene sequence for undesired sequences generated by these codon replacements (e.g., “ATTTA” sequences, inadvertent creation of intron splice recognition sites, unwanted restriction enzyme sites, etc.) and substitute codons that eliminate these sequences.

6. Assemble synthetic gene segments and test for improved expression.

In accordance with this invention, it has been found that the use of alternative codons encoding the same protein sequence may remove the constraints on expression of HPV proteins by human cells.

These methods were used to create the following synthetic gene segments for various papillomavirus genes creating a gene comprised entirely of codons modified for high level expression. While the above procedure provides a summary of our methodology for designing codon modified genes for DNA vaccines, it is understood by one skilled in the art that similar efficacy or increased expression of genes may be achieved by minor variations in the procedure or by minor variations in the sequence.

The expression and detection of HPV proteins in transfected mammalian cells such as HeLa, 293-HEK, or C33A cells has often proved difficult and so for biochemical and immunological studies requiring detectable expression of proteins, or quantities of pure proteins.

The DNA code has 4 letters (A, T, C and G) and uses these to spell three letter “codons” which represent the amino acids of the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several arc coded for by four or more different codons.

Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria and mammalian cells, and some species show a stronger bias away from a random codon selection than others. For these reasons, there is a significant probability that a mammalian gene expressed in E. coli or a viral gene expressed in mammalian cells will have an inappropriate distribution of codons for efficient expression.

There are several examples where changing codons from those which are rare in the host to those which are host-preferred (“codon optimization”) has enhanced heterologous expression levels, for example the BPV (bovine papilloma virus) late genes L1 and L2 have been codon optimized for mammalian codon usage patterns and this has been shown to give increased expression levels over the wild-type HPV sequences in mammalian (Cos-1) cell culture (Zhou et. al. J. Virol 1999. 73, 4972 4982). In this work, every BPV codon which occurred more than twice as frequently in BPV than in mammals (ration of usage>2), and most codons with a usage ratio of >1.5 were conservatively replaced by the preferentially used mammalian codon. In WO97/31115, WO97/48370 and WO98/34640 (Merck & Co., Inc.) codon optimization of HIV genes or segments thereof has been shown to result in increased protein expression and improved immunogenicity when the codon optimised sequences are used as DNA vaccines in the host mammal for which the optimization was tailored.

However, codon optimization does not always result in increased or maximal protein expression. One explanation is that the cell has a defense mechanism that recognizes foreign codon usage. Thus, it is not necessarily similarity to host codon usage, but differentiation from wild-type transfected gene codon usage that may result in increased protein expression. Here, it has been shown that various cell lines were not able to efficiently express E6 protein despite codon optimization to the host cell.

According to a first aspect, the present invention provides a polynucleotide sequence which encodes an HPV amino acid sequence, wherein the codon usage pattern of the polynucleotide sequence differentiates from the wild-type sequence. The polynucleotide sequence may be a DNA sequence, for example a double stranded DNA sequence. Preferably the polynucleotide sequence encodes a HPV E6 polypeptide of an HPV type or sub-type associated with cervical cancer, benign cutaneous warts or genital warts, for example types, 1 4, 6, 7, 10, 11, 16, 18, 26 29, 31, 33, 35, 39, 45, 51, 52, 56, 58, 59, and 68, preferably types 6, 11, 16, 18, 33 or 45, which are associated particularly with cervical cancer and genital warts.

Accordingly, there is provided a synthetic gene comprising a plurality of codons together encoding an HPV amino acid sequence, wherein the selection of the possible codons used for encoding the amino acid sequence has been changed to differentiate from the native sequence. The sequence may be differentiated by 10%, 15%, 20% or 25% or greater. Alternatively, the codons can be modified so that 5%, 10%, 15%, or 20% or greater of the codons have been altered. In another embodiment, the codon sequence may be modified such that 5%, 10%, 15%, 20%, 25%, 30%, 50%, 60%, 70%, 80%, 90%, 95%, 99% or greater of the codons of the gene have the maximum number of nucleotide changes such that there is still degeneracy and the same amino acid is encoded. For example, for cysteine, the maximum number of nucleotide changes is 1, since only the 3^{rd} position may be altered while still coding for the same amino acid (e.g. the codon UGU can be changed to UGC). As another example, for leucine, the maximum number of nucleotide changes is two while retaining coding for the amino acid leucine (e.g. UUA can be modified to CUC). As another example, for serine, the maximum number of nucleotide changes is three while retaining coding for the amino acid leucine (e.g. the codon UCU can be changed to AGC which both code for serine). In another embodiment, the codon sequence can be modified by having 5%, 10%, 15%, 20%, 25% or greater of the codons modified to have a ration of usage <1. In yet another embodiment, the codon sequence can be modified by modifying 5%, 10%, 15%, 20%, 25%, 30%, 50%, 60%, 70%, 80%, 90%, 95%, 99% or greater of the codons to have a ration of usage <1.

In certain embodiments, the encoded amino acid sequence is a mutated HPV amino acid sequence comprising the wild-type sequence with amino acid changes, for example amino acid point mutations, sufficient to reduce or inactivate one or more of the natural biological functions of the polypeptide. The mutated amino acid sequence will desirably retain the immunogenicity of the wild-type polypeptide. The mutated amino acid may have some amino acid modifications from the parent polypeptide sequence such that the codon modified polypeptide that is produced has 90%, 91%, 92%, 93%, 94%, 95%, 96%, 97%, 98%, or 99% identity to the parent sequence.

The codon-modified genes are then assembled into an expression cassette which comprises sequences designed to provide for efficient expression of the protein in a human cell. The cassette preferably contains the codon-modified gene, with related transcriptional and translations control sequences operatively linked to it, such as a promoter, and termination sequences. In a preferred embodiment, the promoter is the cytomegalovirus promoter with the intron A sequence (CMV-intA), although those skilled in the art will recognize that any of a number of other known promoters such as the strong immunoglobulin, or other eukaryotic gene promoters may be used. A preferred transcriptional terminator is the bovine growth hormone terminator, although other known transcriptional terminators may also be used. The combination of CMVintA-BGH terminator is particularly preferred.

According to a second aspect of the invention, an expression vector is provided which comprises and is capable of directing the expression of a polynucleotide sequence according to the first aspect of the invention, encoding an HPV amino acid sequence wherein the codon usage pattern of the polynucleotide sequence is highly diverged from the wildtype sequence but maintains the degeneracy to code for the same polypeptide. The vector may be suitable for driving expression of heterologous DNA in bacterial insect or mammalian cells, particularly human cells. In one embodiment, the expression vector is pmkit-HA.

According to a third aspect of the invention, a host cell may comprise a polynucleotide sequence having codon modification according to the first aspect of the invention, or an expression vector according the second aspect. The host cell may be bacterial, e.g. E. coli, mammalian, e.g. human, or may be an insect cell. Mammalian cells comprising a vector according to the present invention may be cultured cells transfected in vitro or may be transfected in vivo by administration of the vector to the mammal.

In a fourth aspect, the present invention provides a pharmaceutical composition comprising a polynucleotide sequence according to the first aspect of the invention. Preferably the composition comprises a DNA vector according to the second aspect of the present invention. In preferred embodiments the composition comprises a plurality of particles, preferably gold particles, coated with DNA comprising a vector encoding a polynucleotide sequence which encodes an HPV amino acid sequence, wherein the codon usage pattern of the polynucleotide sequence is highly diverged from the wildtype sequence but maintains the degeneracy to code for the same polypeptide. In alternative embodiments, the composition comprises a pharmaceutically acceptable excipient and a DNA vector according to the second aspect of the present invention. The composition may also include an adjuvant.

In a further aspect, the present invention provides a method of making a pharmaceutical composition including the step of altering the codon usage pattern of a wild-type HPV nucleotide sequence, or creating a polynucleotide sequence synthetically, to produce a sequence having a codon usage pattern is highly diverged from the wildtype sequence but maintains the degeneracy to code for the same polypeptide and encoding a codon modified HPV E6 sequence or a sequence having 70%, 80%, 90%, 95%, 96%, 97%, 98% or 99% identity, or an HPV E6 sequence coding for an HPV E6 polypeptide having 1, 2, 3, 4, 5, 6, 7, 8, 9, or 10 or fewer amino acid modifications from the wildtype sequence.

Also provided are the use of a polynucleotide according to the first aspect, or of a vector according to a second aspect of the invention, in the treatment or prophylaxis of an HP V infection, preferably an infection by an HPV type or sub-type associated with cervical cancer, benign cutaneous warts or genital warts, for example types, 1 4, 6, 7, 10, 11, 16, 18, 26 29, 31, 33, 35, 39, 45, 51, 52, 56, 58, 59, and 68. In certain embodiments, the invention provides the use of a polynucleotide according to the first aspect, or of a vector according to a second aspect of the invention, in the treatment or prophylaxis of an HPV infection of type 6, 11, 16, 18, 33 or 45, which are associated particularly with cervical cancer and genital warts, most preferably HPV 11, 6a or 6b. The invention also provides the use of a polynucleotide according to the first aspect, a vector according to the second aspect of the invention or a pharmaceutical composition according to the fourth aspect of the invention, in the treatment or prophylaxis of cutaneous (skin) warts, genital warts, atypical squamous cells of undetermined significance (ASCUS), cervical dysplasia, cervical intraepithelial neoplasia (CIN) or cervical cancer. Accordingly, the present invention also provides the use of a polynucleotide according to the first aspect, or of a vector according to the second aspect of the invention in making a medicament for the treatment or prophylaxis of an HPV infection of any one or more of types 1 4, 6, 7, 10, 11, 16, 18, 26 29, 31, 33, 35, 39, 45, 51, 52, 56, 58, 59, and 68, or any symptoms or disease associated therewith.

The present invention also provides methods of treating or preventing HPV infections, particularly infections by any one or more of HPV types 1 4, 6, 7, 10, 11, 16, 18, 26 29, 31, 33, 35, 39, 45, 51, 52, 56, 58, 59, and 68, or any symptoms or diseases associated therewith, comprising administering an effective amount of a polynucleotide according to the first aspect, a vector according to the second aspect or a pharmaceutical composition according to the fourth aspect of the invention. Administration of a pharmaceutical composition may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence.

As discussed above, the present invention includes expression vectors that comprise the nucleotide sequences of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2.sup.nd Edition. CSH Laboratory Press. (1989).

Preferably, a polynucleotide of the invention, or for use in the invention in a vector, is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e.g. BAC, PAC, YAC), virus or phage vectors provided with a origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell e.g. for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the .beta.-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells or in bacteria may be employed in order to produce quantities of the HPV protein encoded by the polynucleotides of the present invention, for example for use as subunit vaccines or in immunoassays.

The polynucleotides according to the invention have utility in the production by expression of the encoded proteins, which expression may take place in vitro, in vivo or ex vivo. The nucleotides may therefore be involved in recombinant protein synthesis, for example to increase expression yields, or used to screen for therapeutic or diagnostic candidate agents. Where the polynucleotides of the present invention are used in the production of the encoded proteins in vitro or ex vivo, cells, for example in cell culture, will be modified to include the polynucleotide to be expressed. Such cells include transient, or preferably stable mammalian cell lines. Particular examples of cells which may be modified by insertion of vectors encoding for a polypeptide according to the invention include mammalian C33A, HEK293T, CHO, HeLa, 293 and COS cells. Preferably the cell line selected will be one which is not only stable, but also allows for mature glycosylation and cell surface expression of a polypeptide. A polypeptide may be expressed from a polynucleotide of the present invention, in cells of a transgenic non-human animal, preferably a mouse. A transgenic non-human animal expressing a polypeptide from a polynucleotide of the invention is included within the scope of the invention.

Where the polynucleotides of the present invention find use as therapeutic agents, e.g. in DNA vaccination, the nucleic acid will be administered to the mammal e.g. human to be vaccinated. The nucleic acid, such as RNA or DNA, preferably DNA, is provided in the form of a vector, such as those described above, which may be expressed in the cells of the mammal The polynucleotides may be administered by any available technique. For example, the nucleic acid may be introduced by needle injection, preferably intradermally, subcutaneously or intramuscularly. Alternatively, the nucleic acid may be delivered directly into the skin using a nucleic acid delivery device such as particle-mediated DNA delivery (PMDD). In this method, inert particles (such as gold beads) are coated with a nucleic acid, and are accelerated at speeds sufficient to enable them to penetrate a surface of a recipient (e.g. skin), for example by means of discharge under high pressure from a projecting device. (Particles coated with a nucleic acid molecule of the present invention are within the scope of the present invention, as are delivery devices loaded with such particles). The composition desirably comprises gold particles having an average diameter of 0.55 .mu.m, preferably about 2 .mu.m. In preferred embodiments, the coated gold beads are loaded into tubing to serve as cartridges such that each cartridge contains 0.11 mg, preferably 0.5 mg gold coated with 0.15 μg, preferably about 0.5 μg DNA/cartridge.

Suitable techniques for introducing the naked polynucleotide or vector into a patient include topical application with an appropriate vehicle. The nucleic acid may be administered topically to the skin, or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in an amount in the range of 1 pg to 1 mg, preferably 1 pg to 10 μg nucleic acid for particle mediated gene delivery and 10 μg to 1 mg for other routes.

A nucleic acid sequence of the present invention may also be administered by means of specialised delivery vectors useful in gene therapy. Gene therapy approaches are discussed for example by Verme et al, Nature 1997, 389:239 242. Both viral and non-viral vector systems can be used. Viral based systems include retroviral, lentiviral, adenoviral, adeno-associated viral, herpes viral, Canarypox and vaccinia-viral based systems. Non-viral based systems include direct administration of nucleic acids, microsphere encapsulation technology (poly(lactide-co-glycolide) and, liposome-based systems. Viral and non-viral delivery systems may be combined where it is desirable to provide booster injections after an initial vaccination, for example an initial “prime” DNA vaccination using a non-viral vector such as a plasmid followed by one or more “boost” vaccinations using a viral vector or non-viral based system.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e.g. grafting, micro-injection, etc.)

Suitable cells include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumour, e.g. anti-cervical carcinoma effects per se and/or to be immunologically compatible with the receiver (i.e., matched HLA haplotype). APCs may generally be isolated from any of a variety of biological fluids and organs, including tumour and peri-tumoural tissues, and may be autologous, allogeneic, syngeneic or xenogeneic cells.

Certain preferred embodiments of the present invention use dendritic cells or progenitors thereof as antigen-presenting cells, either for transformation in vitro and return to the patient or as the in vivo target of nucleotides delivered in the vaccine, for example by particle mediated DNA delivery. Dendritic cells are highly potent APCs (Banchereau and Steinman, Nature 392:245 251, 1998) and have been shown to be effective as a physiological adjuvant for eliciting prophylactic or therapeutic antitumour immunity (see Timmerman and Levy, Ann. Rev. Med. 50:507 529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, for example the antigen(s) encoded in the constructs of the invention, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594 600, 1998).

Dendritic cells and progenitors may be obtained from peripheral blood, bone marrow, tumour-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF, CD40 ligand, lipopolysaccharide LPS, flt3 ligand (a cytokine important in the generation of professional antigen presenting cells, particularly dentritic cells) and/or other compound(s) that induce differentiation, maturation and proliferation of dendritic cells.

APCs may generally be transfected with a polynucleotide encoding an antigenic HPV amino acid sequence, such as a codon-optimised polynucleotide as envisaged in the present invention. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the particle mediated approach described by Mahvi et al., Immunology and cell Biology 75:456 460, 1997.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use. Vaccines comprising nucleotide sequences intended for administration via particle mediated delivery may be presented as cartridges suitable for use with a compressed gas delivery instrument, in which case the cartridges may consist of hollow tubes the inner surface of which is coated with particles bearing the vaccine nucleotide sequence, optionally in the presence of other pharmaceutically acceptable ingredients.

The pharmaceutical compositions of the present invention may include adjuvant compounds, or other substances which may serve to modulate or increase the immune response induced by the protein which is encoded by the DNA. These may be encoded by the DNA, either separately from or as a fusion with the antigen, or may be included as non-DNA elements of the formulation. Examples of adjuvant-type substances which may be included in the formulations of the present invention include ubiquitin, lysosomal associated membrane protein (LAMP), hepatitis B virus core antigen, flt3-ligand and other cytokines such as IFN-.gamma. and GMCSF.

Other suitable adjuvants are commercially available such as, for example, Freund's Incomplete Adjuvant and Complete Adjuvant (Difco Laboratories, Detroit, Mich.); Imiquimod (3M, St. Paul, Minn.); Resimiquimod (3M, St. Paul, Minn.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

In the formulations of the invention it is preferred that the adjuvant composition induces an immune response predominantly of the Th1 type. Thus the adjuvant may serve to modulate the immune response generated in response to the DNA-encoded antigens from a predominantly Th2 to a predominantly Th1 type response. High levels of Th1-type cytokines (e.g., IFN-, TNF, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145 173, 1989.

Accordingly, suitable adjuvants for use in eliciting a predominantly Th 1-type response include, for example, a combination of monophosphoryl lipid A, preferably 3-de-O-acylated monophosphoryl lipid A (3D-MPL) together with an aluminium salt. Other known adjuvants which preferentially induce a TH1 type immune response include CpG containing oligonucleotides. The oligonucleotides are characterised in that the CpG dinucleotide is unmethylated. Such oligonucleotides are well known and are described in, for example WO96/02555 Immunostimulatory DNA sequences are also described, for example, by Sato et al., Science 273:352, 1996. CpG-containing oligonucleotides may be encoded separately from the papilloma antigen(s) in the same or a different polynucleotide construct, or may be immediately adjacent thereto, e.g. as a fusion therewith. Alternatively the CpG-containing oligonucleotides may be administered separately i.e. not as part of the composition which includes the encoded antigen. CpG oligonucleotides may be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol.

Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Other preferred adjuvants include adjuvant molecules of the general formula (I) HO(CH2CH2O)n-A-R Formula (I): wherein, n is 1 50, A is a bond or —C(O)—, R is C1 50 alkyl or Phenyl C1 50 alkyl.

One embodiment of the present invention consists of a formulation comprising a polyoxyethylene ether of general formula (I), wherein n is between 1 and 50, preferably 424, most preferably 9; the R component is C1 50, preferably C4-C20 alkyl and most preferably C12 alkyl, and A is a bond. The concentration of the polyoxyethylene ethers should be in the range 0.120%, preferably from 0.110%, and most preferably in the range 0.11%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12th edition: entry 7717). These adjuvant molecules are described in WO 99/52549. The polyoxyethylene ether according to the general formula (I) above may, if desired, be combined with another adjuvant. For example, a preferred adjuvant combination is preferably with CpG as described in the pending UK patent application GB 9820956.2.

Where the vaccine includes an adjuvant, the vaccine formulation may be administered in two parts. For example, the part of the formulation containing the nucleotide construct which encodes the antigen may be administered first, e.g. by subcutaneous or intramuscular injection, or by intradermal particle-mediated delivery, then the part of the formulation containing the adjuvant may be administered subsequently, either immediately or after a suitable time period which will be apparent to the physician skilled in the vaccines arts. Under these circumstances the adjuvant may be administered by the same route as the antigenic formulation or by an alternate route. In other embodiments the adjuvant part of the formulation will be administered before the antigenic part. In one embodiment, the adjuvant is administered as a topical formulation, applied to the skin at the site of particle mediated delivery of the nucleotide sequences which encode the antigen(s), either before or after the particle mediated delivery thereof.

Historically, vaccines have been seen as a way to prevent infection by a pathogen, priming the immune system to recognise the pathogen and neutralise it should an infection occur. The vaccine includes one or more antigens from the pathogen, commonly the entire organism, either killed or in a weakened (attenuated) form, or selected antigenic peptides from the organism. When the immune system is exposed to the antigen(s), cells are generated which retain an immunological “memory” of it for the lifetime of the individual. Subsequent exposure to the same antigen (e.g. upon infection by the pathogen) stimulates a specific immune response which results in elimination or inactivation of the infectious agent.

There are two arms to the immune response: a humoral (antibody) response and a cell-mediated response. Protein antigens derived from pathogens that replicate intracellularly (viruses and some bacteria) are processed within the infected host cell releasing short peptides which are subsequently displayed on the infected cell surface in association with class I major histocompatability (MHC I) molecules. When this associated complex of MHC I and peptide is contacted by antigen-specific CD8+ T-cells the T-cell is activated, acquiring cytotoxic activity. These cytotoxic T-cells (CTLs) can lyse infected host cells, so limiting the replication and spread of the infecting pathogen. Another important arm of the immune response is controlled by CD4+ T-cells. When antigen derived from pathogens is released into the extracellular milieu they may be taken up by specialised antigen-presenting cells (APCs) and displayed upon the surface of these cells in association with MHC II molecules. Recognition of antigen in this complex stimulates CD4+ T-cells to secrete soluble factors (cytokines) which regulate the effector mechanisms of other T-cells. Antibody is produced by B-cells. Binding of antigen to secreted antibody may neutralise the infectivity of a pathogen and binding of antigen to membrane-bound antibody on the surface of B-cells stimulates division of the B-cell so amplifying the B-cell response. In general, both antibody and cell-mediated immune responses (CD8+ and CD4+) are required to control infections by pathogens.

It is believed that it may be possible to harness the immune system, even after infection by a pathogen, to control or resolve the infection by inactivation or elimination of the pathogen. Such immune therapies (also known as “therapeutic” vaccines or immunotherapeutics) would ideally require a cell-mediated response to be effective, although both humoral and cell-mediated immune responses may be evoked.

It has been demonstrated (Benvenisty, N and Reshaf, L. PNAS 83 955-9555) that inoculation of mice with calcium phosphate precipitated DNA results in expression of the peptides encoded by the DNA. Subsequently, intramuscular injection into mice of plasmid DNA which had not been precipitated was shown to result in uptake of the DNA into the muscle cells and expression of the encoded protein. Because expression of the DNA results in production of the encoded pathogen proteins within the host's cells, as in a natural infection, this mechanism can stimulate the cell-mediated immune response required for immune therapies or therapeutic vaccination, so a DNA-based drug could be applied as a prophylactic vaccine or as an immune therapy. DNA vaccines are described in WO90/11092 (Vical, Inc.). DNA vaccination may be delivered by mechanisms other than intra-muscular injection. For example, delivery into the skin takes advantage of the fact that immune mechanisms are highly active in tissues that are barriers to infection such as skin and mucous membranes. Delivery into skin could be via injection, via jet injector (which forces a liquid into the skin, or underlying tissues including muscles, under pressure) or via particle bombardment, in which the DNA may be coated onto particles of sufficient density to penetrate the epithelium (U.S. Pat. No. 5,371,015). For example, the nucleotide sequences may be incorporated into a plasmid which is coated on to gold beads which are then administered under high pressure into the epidermis, such as, for example, as described in Haynes et al J. Biotechnology 44: 37 42 (1996). Projection of these particles into the skin results in direct transfection of both epidermal cells and epidermal Langerhan cells. Langerhan cells are antigen presenting cells (APC) which take up the DNA, express the encoded peptides, and process these for display on cell surface MHC proteins. Transfected Langerhan cells migrate to the lymph nodes where they present the displayed antigen fragments to lymphocytes, evoking an immune response. Very small amounts of DNA (less than 1 μg, often less than 0.5 μg) are required to induce an immune response via particle mediated delivery into skin and this contrasts with the milligram quantities of DNA known to be required to generate immune responses subsequent to direct intramuscular injection.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Generation of Codon Optimized and Modified HPV35 E6 Sequences

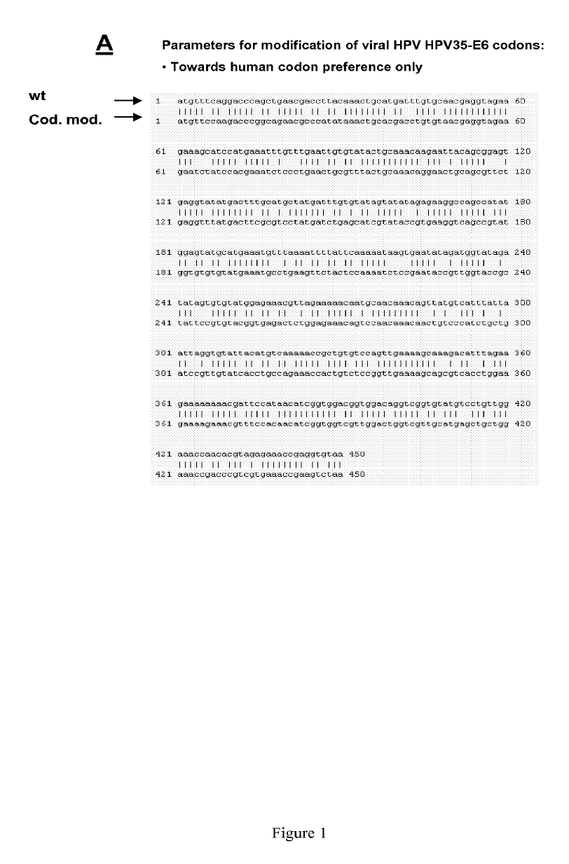

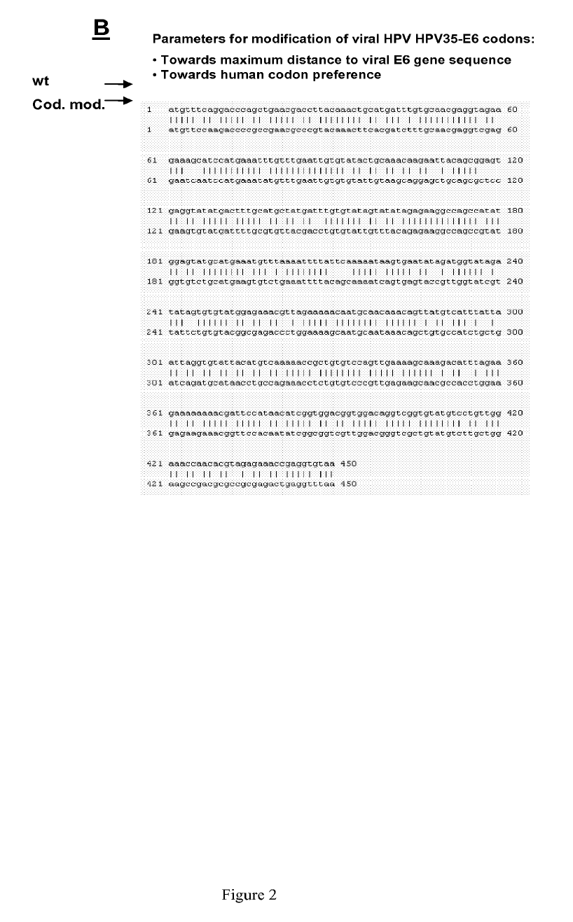

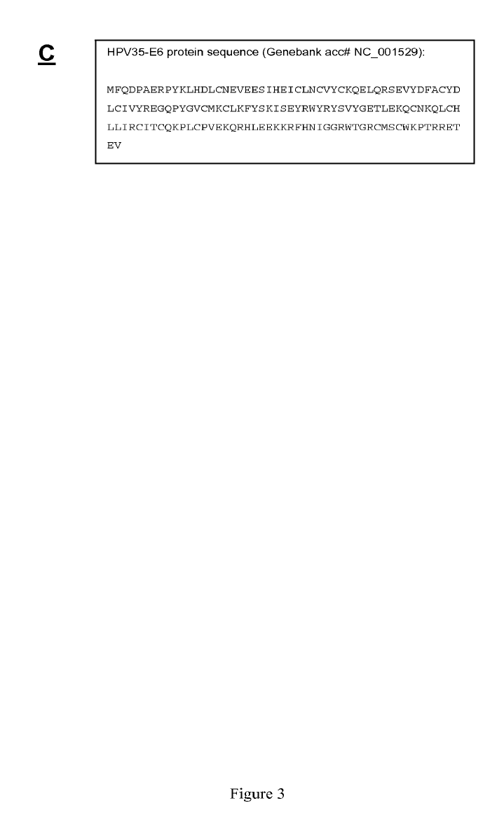

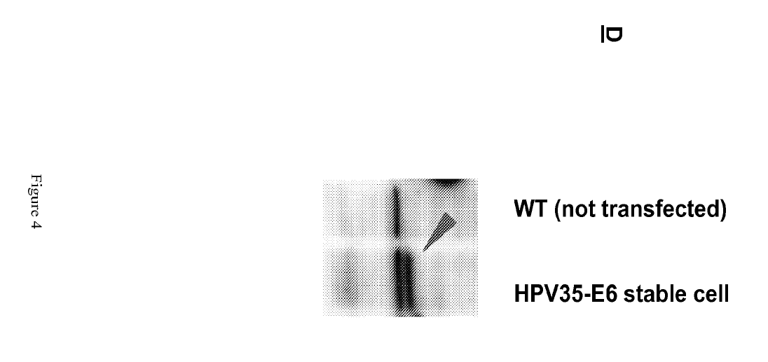

A wild-type coding sequence of HPV35 E6 protein is shown as the wild-type (wt) sequence in FIGS. 1 and 2. The HPV35 E6 sequence is sent to a laboratory (DNA2.0, Menlo Park, Calif.) for modification of sequence. The HPV35 E6 nucleotide sequence is modified to maximize codon usage with human preference and the sequence is shown in FIG. 1 as codon optimized. A comparison of the wild-type and codon optimized sequences is aligned in FIG. 1. Neither the wild type sequence nor the codon optimized sequences, when transfected into a C33A cell line is visible by Western blot (data not shown). The HPV35 E6 nucleotide sequence is also modified to maximize the number of changes in the sequence while retaining degeneracy for coding the HPV35 E6 protein. The resulting codon modified sequence is shown in FIG. 2, where it is aligned with wild-type. Both the codon optimized and codon modified sequences generated code for the same HPV35 E6 protein shown in FIG. 3. FIG. 4 shows a Western blot comparison of wild-type transfected and codon modified transfected cell lines. Only the codon modified sequence shows protein expression.

What is claimed is:

1. A method comprising modifying a nucleic acid molecule, wherein wherein the nucleic acid molecule codes for human papilloma virus E6, and further wherein the nucleic acid molecule comprises a sequence of nucleotides that is codon-modified for high level expression in a host cell, the nucleic acid molecule has been modified such that at least 10% of the codons have been modified, and the nucleic acid molecule has been modified such that at least 5% of the codons have been modified to have a ration of usage less than 1.

2. The method of claim 1, wherein the nucleic acid molecule has been modified by at least 10% from the native sequence.

3. The method of claim 1, wherein the nucleic acid molecule has been modified such that at least 10% of the codons have been modified.

4. The method of claim 1, wherein the nucleic acid molecule has been modified such that at least 5% of the codons have the maximum number of changes such that there is still degeneracy for the amino acid originally encoded.

5. The method of claim 1, wherein the nucleic acid molecule has been modified such that at least 5% of the codons have been modified to have a ration of usage less than 1.

6. The method of claim 1, wherein the nucleic acid molecule codes for human papilloma virus E6.

7. A method comprising the steps of:
a) Modifying a nucleic acid molecule, wherein the nucleic acid molecule comprises a sequence of nucleotides that is codon-modified for high level expression in a host cell;
b) transforming a host cell with the nucleic acid molecule; and
c) cultivating the transformed cell under conditions that permit expression of the nucleic acid molecule to produce a protein product.

8. The method of claim 7, wherein the nucleic acid molecule has been modified by at least 10% from the native sequence.

9. The method of claim 7, wherein the nucleic acid molecule has been modified such that at least 10% of the codons have been modified.

10. The method of claim 7, wherein the nucleic acid molecule has been modified such that at least 5% of the codons have the maximum number of changes such that there is still degeneracy for the amino acid originally encoded.

11. The method of claim 7, wherein the nucleic acid molecule has been modified such that at least 5% of the codons have been modified to have a ration of usage less than 1.

12. The method of claim 7, wherein the nucleic acid molecule codes for human papilloma virus E6.

13. The method of claim 7, wherein the host cell is a 293-HEK or C33A cell.

14. A composition comprising a modified nucleic acid molecule.

15. The composition of claim 14, wherein the nucleic acid molecule has been modified by at least 10% from the native sequence.

16. The composition of claim 14, wherein the nucleic acid molecule has been modified such that at least 10% of the codons have been modified.

17. The composition of claim 14, wherein the nucleic acid molecule has been modified such that at least 5% of the codons have the maximum number of changes such that there is still degeneracy for the amino acid originally encoded.

18. The composition of claim 14, wherein the nucleic acid molecule has been modified such that at least 5% of the codons have been modified to have a ration of usage less than 1.


SEQUENCE LISTING (4 sequences)

SEQ ID NO: 1 - DNA, 450 bp, Human papillomavirus
atgtttcaggacccagctgaacgaccttacaaactgcatgatttgtgcaacgaggtagaagaaagcatccatgaaatttgtttgaattgtgtatactgcaaacaagaattacagcggagtgaggtatatgactttgcatgctatgatttgtgtatagtatatagagaaggccagccatatggagtatgcatgaaatgtttaaaattttattcaaaaataagtgaatatagatggtatagatatagtgtgtatggagaaacgttagaaaaacaatgcaacaaacagttatgtcatttattaattaggtgtattacatgtcaaaaaccgctgtgtccagttgaaaagcaaagacatttagaagaaaaaaaacgattccataacatcggtggacggtggacaggtcggtgtatgtcctgttggaaaccaacacgtagagaaaccgaggtgtaa

SEQ ID NO: 2 - DNA, 450 bp, Artificial Sequence
  Description of Artificial Sequence Synthetic polynucleotide
atgttccaagacccggcagaacgcccatataaactgcacgacctgtgtaacgaggtagaagaatctatccacgaaatctccctgaactgcgtttactgcaaacaggaactgcagcgttctgaggtttatgacttcgcgtcctatgatctgagcatcgtataccgtgaaggtcagccgtatggtgtgtgtatgaaatgcctgaagttctactccaaaatctccgaataccgttggtaccgctattccgtgtacggtgagactctggagaaacagtccaacaaacaactgtcccatctgctgatccgttgtatcacctgccagaaaccactgtctccggttgaaaagcagcgtcacctggaagaaaagaaacgtttccacaacatcggtggtcgttggactggtcgttgcatgagctgctggaaaccgacccgtcgtgaaaccgaagtctaa

SEQ ID NO: 3 - DNA, 450 bp, Artificial Sequence
  Description of Artificial Sequence Synthetic polynucleotide
atgttccaagaccccgccgaacgcccgtacaaacttcacgatctttgcaacgaggtcgaggaatcaatccatgaaatatgtttgaattgtgtgtattgtaagcaggagctgcagcgctccgaagtgtatgattttgcgtgttacgacctgtgtattgtttacagagaaggccagccgtatggtgtctgcatgaagtgtctgaaattttacagcaaaatcagtgagtaccgttggtatcgttattctgtgtacggcgagaccctggaaaagcaatgcaataaacagctgtgccatctgctgatcagatgcataacctgccagaaacctctgtgtcccgttgagaagcaacgccacctggaagagaagaaacggttccacaatatcggcggtcgttggacgggtcgctgtatgtcttgctggaagccgacgcgccgcgagactgaggtttaa

SEQ ID NO: 4 - protein, 149 aa, Artificial Sequence
  Description of Artificial Sequence Synthetic polypeptide
MFQDPAERPYKLHDLCNEVEESIHEICLNCVYCKQELQRSEVYDFACYDLCIVYREGQPYGVCMKCLKFYSKISEYRWYRYSVYGETLEKQCNKQLCHLLIRCITCQKPLCPVEKQRHLEEKKRFHNIGGRWTGRCMSCWKPTRRETEV